(19) United States
(12) Patent Application Publication
(10) Pub. No.: US 2005/0266034 A1
(43) Pub. Date: Dec. 1, 2005

(54) Cosmetic and/or dermatological composition based on at least one surfactant, at least one monocarboxylic acid, and at least one polyol

(76) Inventors: Rainer Muller, Leopoldshafen (DE); Thomas Hippe, Fourqueux (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/125,221

(22) Filed: May 10, 2005

Related U.S. Application Data
(60) Provisional application No. 60/580,366, filed on Jun. 18, 2004.

(30) Foreign Application Priority Data
May 10, 2004 (FR) 04 05031

(51) Int. Cl. A61K 7/00

(52) U.S. Cl. 424/401

(57) ABSTRACT

Disclosed herein is a cosmetic and/or dermatological composition, such as a haircare composition, wherein the composition comprises, in a physiologically acceptable medium, at least one surfactant; at least one monocarboxylic acid chosen from benzoic acid, salicylic acid, sorbic acid, dehydroacetic acid, propionic acid, caproic acid, or and salts thereof, and present in the composition in an amount ranging from 0.1% to 1% by weight, relative to the total weight of the composition; at least one polyol having a molecular mass of less than 500, present in the composition in an amount ranging from 1 % to 20% by weight relative to the total weight of the composition; wherein the water activity at 25° C. of the composition is less than or equal to 0.96.

This application claims benefit of U.S. Provisional Application No. 60/580,366, filed Jun. 18, 2004, the contents of which are incorporated herein by reference. This application also claims benefit of priority under 35 U.S.C. § 119 to French Patent Application No. 04 05031, filed May 10, 2004, the contents of which are also incorporated by reference.

Disclosed herein are novel cosmetic and/or dermatological compositions, such as haircare compositions, comprising at least one surfactant, at least one monocarboxylic acid, and at least one polyol. Also disclosed herein is the use of these compositions in cosmetics, and the use of these compositions for preparing a dermatological composition for treating disorders associated with Aspergillus niger.

Cosmetic compositions that contain a large amount of water, such as shampoos, should contain preserving agents to prevent the growth of microorganisms. For example, molds, such as Aspergillus niger, may be difficult to eliminate. Thus, high concentrations of preserving agents may be necessary.

However, preserving agents occasionally have an allergic potential. It consequently may be desirable to reduce the concentration of preserving agents.

It thus appears to be desirable to have available compositions that can prevent the growth of microorganisms, while at the same time having a reduced concentration of preserving agents.

European Patent No. EP 1 325 731 discloses a microbicidal agent comprising a combination of anisic acid with a polyol and a cationic surfactant.

However, these compositions based on anisic acid may be relatively unstable.

Russian Federation Patent No. RU 2 020 99 describes compositions based on surfactant, glycerol, NaCl, and salicylic acid.

However, these compositions, having a water activity at 25° C. of greater than 0.96, may have insufficient antimicrobial activity.

The present inventors have discovered, surprisingly, that compositions comprising at least one surfactant, at least one particular monocarboxylic acid, and at least one. polyol, with a water activity at 25° C. of less than 0.96, and/or with a polyol content ranging from 3% to 20% by weight, may have sufficient activity against microorganisms.

The present inventors have also discovered that shampoos based on this composition may have unchanged properties, such as unchanged physicochemical and cosmetic properties, for example the viscosity and the foam quality.

Finally, the present inventors have also discovered that these compositions may have reinforced activity against Aspergillus niger, and thus make it possible to prepare shampoos having an attractive and healthy appearance, while at the same time having the ability to combat disorders related to Aspergillus niger, such as scalp irritations and diseases related to this microorganism.

One embodiment disclosed herein is thus a cosmetic and/or dermatological composition, such as a haircare composition, comprising, in a physiologically acceptable medium:
a) at least one surfactant;
b) at least one monocarboxylic acid chosen from benzoic acid, salicylic acid, sorbic acid, dehydroacetic acid, propionic acid, caproic acid, and salts thereof, and present in the composition in an amount ranging from 0.1% to 1% by weight relative to the total weight of the composition; and

c) at least one polyol, which may be non-etherified, having a molecular mass of less than 500, present in the composition in an amount ranging from 1% to 20% by weight relative to the total weight of the composition; the water activity at 25° C. of the composition being less than or equal to 0.96.

Another embodiment disclosed herein is a cosmetic and/or dermatological composition, such as a haircare composition, comprising, in a physiologically acceptable medium:
a) at least one surfactant;
b) at least one monocarboxylic acid chosen from benzoic acid, salicylic acid, sorbic acid, dehydroacetic acid, propionic acid, caproic acid, and salts thereof, and present in the composition in an amount ranging from 0.1% to 1% by weight relative to the total weight of the composition, and
c) at least one polyol, which may be non-etherified, having a molecular mass of less than 500, present in the composition in an amount ranging from 3% to 20% by weight, such as from 4% to 20% by weight relative to the total weight of the composition.

Another embodiment relates to a cosmetic haircare process using the compositions disclosed herein.

Another embodiment is the use of the compositions disclosed herein for the cosmetic treatment of the hair and the scalp.

Yet another embodiment is the use of the compositions disclosed herein for the preparation of a dermatological composition for treating disorders related to Aspergillus niger.

Other characteristics, aspects, subjects, and advantages of the embodiments disclosed herein will emerge even more clearly on reading the description and the examples that follow.

The compositions disclosed herein comprise at least one particular monocarboxylic acid, or a mineral or organic salt thereof.

In certain embodiments, the at least one monocarboxylic acid is chosen from benzoic acid, salicylic acid, sorbic acid, and dehydroacetic acid.

In certain embodiments, the at least one monocarboxylic acid is non-hydroxylated.

The at least one monocarboxylic acid may be present in the composition in an amount ranging from 0.15% to 0.5% by weight relative to the total weight of the composition.

Any polyol with a molecular mass of less than 500 may be used in the compositions disclosed herein. As used herein, the term “polyol” means any compound containing at least two free OH functions. The at least one polyol may be non-etherified.

The at least one polyol may, for example, be chosen from sorbitol, glucose, fructose, xylose, trehalose, sucrose, maltose, lactose, polyethylene glycols, C₃-C₈ diols, and C₃-C₈ triols, such as butanediol.

Non-etherified polyols and non-esterified polyols may be used.

The polyols that may be used include glycerol and 1,2-propylene glycol.

The at least one polyol may be present in the composition in an amount ranging from 1% to 10%, such as from 4% to 10%, by weight relative to the total weight of the composition.

The at least one surfactant may be chosenfrom at least one of anionic, amphoteric, non-ionic, zwitterionic, and cationic surfactants.

The surfactants that may be mentioned include the following:

(i) Anionic surfactants:

By way of example of anionic surfactants that may be used herein, alone or as mixtures, mention may be made (non-limiting list) of salts (such as alkali metal salts, for example sodium salts, ammonium salts, amine salts, amino alcohol salts, and magnesium salts) of the following compounds: alkyl sulphates, alkyl ether sulphates, alkylamido ether sulphates, alkylarylpolyether sulphates, monoglyceride sulphates, alkyl sulphonates, alkyl phosphates, alkylamide sulphonates, alkylaryl sulphonates, α-olefin sulphonates, paraffin sulphonates, (C₆-C₂₄)alkyl sulphosuccinates, (C₆-C₂₄)alkyl ether sulphosuccinates, (C₆-C₂₄)alkylamide sulphosuccinates, (C₆-C₂₄)alkyl sulphoacetates, (C₆-C₂₄)acyl sarcosinates, and (C₆-C₂₄)acyl glutamates. It is also possible to use (C₆-C₂₄)alkylpolyglycoside carboxylic esters such as alkylglucoside citrates, alkylpolyglycoside tartrates, and alkylpolyglycoside sulphosuccinates; alkylsulphosuccinamates; acyl isethionates; and N-acyl taurates, wherein the alkyl or acyl radical of all of these compounds may contain 12 to 20 carbon atoms, and wherein the aryl radical may denote a phenyl or benzyl group. Among the anionic surfactants which may also be used, mention may be made of fatty acid salts such as oleic, ricinoleic, palmitic, and stearic acid salts; coconut oil acid; hydrogenated coconut oil acid; and acyl lactylates in which the acyl radical contains 8 to 20 carbon atoms. It is also possible to use at least one of alkyl D-galactoside uronic acids and their salts, polyoxyalkylenated (C₆-C₂₄)alkyl ether carboxylic acids, polyoxyalkylenated (C₆-C₂₄)alkylaryl ether carboxylic acids, and polyoxyalkylenated (C₆-C₂₄)alkylamido ether carboxylic acids and their salts, such as those containing from 2 to 50 alkylene oxide groups, for example ethylene oxide groups.

(ii) Nonionic surfactants:

The nonionic surfactants are compounds that may be well known per se (see in this respect “Handbook of Surfactants” by M. R. Porter, published by Blackie & Son (Glasgow and London), 1991, pp. 116-178), and their nature generally does not affect the embodiments disclosed herein. Thus, they may be chosen from (non-limiting list) at least one of polyethoxylated or polypropoxylated alkylphenols, alpha-diols, and alcohols, having a fatty chain containing, for example, 8 to 18 carbon atoms, it being possible for the number of ethylene oxide or propylene oxide groups to range from 2 to 50. Mention may also be made of copolymers of ethylene oxide and of propylene oxide; condensates of ethylene oxide and of propylene oxide with fatty alcohols; polyethoxylated fatty amides, which may have from 2 to 30 mol of ethylene oxide; polyglycerolated fatty amides containing on average 1 to 5, such as 1.5 to 4, glycerol groups; oxyethylenated fatty acid esters of sorbitan having from 2 to 30 mol of ethylene oxide; fatty acid esters of sucrose; fatty acid esters of polyethylene glycol; alkylpolyglycosides; N-alkylglucamine derivatives; and amine oxides such as (C₁₀-C₁₄)alkylamine oxides and N-acylaminopropylmorpholine oxides. It will be noted that the alkylpolyglycosides constitute nonionic surfactants that may be mentioned in the context of certain embodiments disclosed herein.

(iii) Amphoteric or zwitterionic surfactants:

The amphoteric or zwitterionic surfactants, the nature of which generally does not affect the embodiments disclosed herien, may be (non-limiting list) aliphatic secondary or tertiary amine derivatives in which the aliphatic radical is a linear or branched chain containing 8 to 18 carbon atoms and containing at least one water-solubilizing anionic group (for example carboxylate, sulphonate, sulphate, phosphate, and phosphonate). Mention may also be made of (C₈-C20)alkylbetaines, sulphobetaines, (C₈-C₂₀)alkylamido (C₁-C₆)alkylbetaines, and (C₈-C₂₀)alkylamido(C₁-C₆)alkylsulphobetaines.

Among the amine derivatives, mention may be made of the products sold under the name Miranol®, as described in U.S. Pat. Nos. 2,528,378 and 2,781,354 and classified in the CTFA dictionary, 3rd edition, 1982, under the names Amphocarboxyglycinates and Amphocarboxypropionates, with the respective structures:
R₂—CONHCH₂CH₂—N(R₃)(R₄)(CH₂COO—)
in which: R₂ is chosen from linear or branched C₅-C₂₀ alkyl radicals of an acid R₂—COOH present in hydrolysed coconut oil heptyl radicals, nonyl radicals, and undecyl radicals, R₃ denotes a beta-hydroxyethyl group, and R₄ denotes a carboxymethyl group; and
R₂—CONHCH₂CH₂—N(B)(C)
in which:

B represents —CH₂CH₂OX′, C represents —(CH₂)_{z}—Y′, wherein z is 1 or 2,

X′ is chosen from —CH₂CH₂—COOH and hydrogen,

Y′ is chosen from —COOH and —CH₂—CHOH—SO₃H,

R₂′ is chosen from linear or branched, saturated or unsaturated, C₅-C₂₀ alkyl radicals of an acid R₂′-COOH present for example in coconut oil and hydrolysed linseed oil; alkyl radicals, such as C₇, C₉, C₁₁ and C₁₃ alkyl radicals, C₁₇ alkyl radicals and its iso form, and unsaturated C₁₇ radicals.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Capryloamphodipropionate, Lauroamphodipropionic acid, and Cocoamphodipropionic acid.

By way of example, mention may be made of the cocoamphodiacetate sold under the trade name Miranol® C2M concentrate by the company Rhodia Chimie.

(iv) Cationic surfactants:

Among the cationic surfactants, mention may be made (non-limiting list) of: salts of optionally polyoxyalkylenated primary, secondary, or tertiary fatty amines; quaternary ammonium salts such as tetraalkylammonium, alkylamidoalkyltrialkylammonium, trialkylbenzylammonium, trialkylhydroxyalkylammonium, alkylpyridinium chlorides, and alkylpyridium bromides; imidazoline derivatives; and cationic amine oxides.

The at least one surfactant may be present in the composition in an amount ranging from 0.01% to 40%, such as from 0.5% to 30% or from 4% to 25%, by weight relative to the total weight of the composition.

According to one embodiment disclosed herein, the composition also comprises at least one additional salt other than the at least one salt of monocarboxylic acid and of the at least one surfactant mentioned above. In this case, the antifungal activity of the composition may be reinforced.

The at least one additional salt may be organic or mineral.

Among the salts that may be mentioned are mineral or C₁-C₇ organic acid salts other than the salts of monocarboxylic acids and the surfactants described above. Mention may also be made of organic salts of ammonium or of a primary amine, which is optionally hydroxylated.

At least one metal salt may also be used. The metal may be chosen from alkali metals, alkaline-earth metals, and transition metals.

The metal may be monovalent or divalent. The metal may be chosen from Na, Ca, Li, K, Mg, Cu, and Zn.

The salt may be chosen from sodium chloride, potassium chloride, lithium chloride, ammonium chloride, sodium sulphate, magnesium sulphate, sodium acetate, copper sulphate, and sodium citrate.

The at least one metal salt may be present in the composition in an amount ranging from 1% to 10%, such as from 1% to 5% or from 1% to 4%, by weight relative to the total weight of the composition.

The salt/polyol mass ratio may range from 1:1 to 1:10.

The water activity at 25° C. of the composition may be less than or equal to 0.95, such as ranging from 0.75 to 0.95. This activity may be measured using the Hygroscop BT-RS1 instrument from the company Rotronic. As used herein, the water activity is the ratio between the vapor pressure of a product and the vapor pressure of pure water, at the same temperature.

The pH of the composition may be less than 7, for example the pH may be less than 6.

The physiologically acceptable medium may comprise water or a mixture of water and at least one physiologically acceptable organic solvent. Solvents that may be mentioned include C₁-C₄ alcohols.

Among these alcohols, ethanol and isopropanol may be mentioned. For example, ethanol may be used.

The concentration of the at least one organic solvent may be less than 30%, such as 10%, by weight relative to the total weight of the composition.

The composition disclosed herein may also contain at least one additive chosen from fragrances; screening agents; preserving agents; proteins; vitamins; nonionic, anionic, cationic, amphoteric, and zwitterionic polymers; mineral, plant, and synthetic oils; and any other additive conventionally used in cosmetic compositions, such as antidandruff agents, agents for preventing hair loss, dyes, pigments, and reducing agents.

These additives may be present in the composition in an amount ranging from 0.001% to 20% by weight relative to the total weight of the composition. The precise amount of each additive depends on its nature and on the chosen haircare application and is readily determined by a person skilled in the art.

Needless to say, a person skilled in the art will take care to select the at least one additive such that the advantageous properties of the composition disclosed herein are not, or are not substantially, adversely affected by the envisaged addition.

The compositions disclosed herein may be in the form of lotions, shampoos, mousses, creams, gels, sticks, sprays, balms, and soaps.

The compositions disclosed herein may be used for manufacturing numerous haircare products, for instance products for fixing and/or holding the hair; conditioning products, such as sheen formulations; and haircare products.

These compositions may be packaged in various forms, such as in pump-dispenser bottles and in aerosol containers, so as to be able to apply the composition in vaporized form or in the form of a mousse.

When a composition as disclosed herein is packaged in aerosol form, it comprises at least one propellant that may be chosen from volatile hydrocarbons such as n-butane, propane, isobutene, pentane, halogenated hydrocarbons, and mixtures thereof. Carbon dioxide, nitrous oxide, dimethyl ether (DME), nitrogen, and compressed air may also be used as propellant. Mixtures of propellants may also be used. Dimethyl ether, for example, may be used.

In certain embodiments, the at least one propellant is present in a concentration ranging from 5% to 90% by weight, such as from 10% to 60% by weight relative to the total weight of the composition in the aerosol device.

One embodiment disclosed herein is also the use of a composition as defined above for the cosmetic treatment of the hair and the scalp.

Another embodiment disclosed herein is also the use of a composition as defined above as an antifungal cosmetic composition.

Further disclosed herein is the use of a composition as defined above for the preparation of a dermatological composition for treating disorders related to Aspergillus niger.

Another embodiment is also a cosmetic treatment process comprising applying the cosmetic composition disclosed herein to wet or dry hair, optionally followed by rinsing.

In one embodiment of the invention, the compositions disclosed herein are used as shampoos for washing and treating the hair and the scalp.

In certain cases, the compositions are applied to the wet or dry hair, in amounts that are sufficient to wash it, this application being followed by rinsing.

The examples that follow are intended to illustrate certain embodiments disclosed herein.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are-reported as precisely as possible. Any numerical value, however, inherently contain certain errors necessarily resulting from the standard deviation found in their respective testing measurements. The following examples are intended to illustrate the invention without limiting the scope as a result.

### EXAMPLE 1

Shampoos of compositions 1 and 2 below were prepared:

| Composition | 1 | 2 |
|---|---|---|
| Sodium lauryl ether sulfate | 15 g | 15 g |
| Containing 70% active material | | |
| Cocamidopropyl betaine | 6.4 g | 6.4 g |
| containing 37% active material | | |
| Polyquaternium-10 | 0.5 g | 0.5 g |
| Sodium chloride | 3.8 g | — |
| Glycerol | 4 g | — |
| Sodium benzoate | 0.3 g | 0.3 g |
| Citric acid | 0.04 g | 0.06 g |
| Water qs | 100 g | 100 g |
| pH | 5.7 | 5.6 |
| Water activity | 0.95 | 0.99 |

| | Number of | |
|---|---|---|
| | microorganisms | |
| | after 7 days | |
| Escherichia coli | <200 | 3.1E + 05 |
| Pseudomonas aeruginosa | <200 | 2.5E + 04 |
| Staphylococcus aureus | <200 | <200 |
| Enterococcus faecalis | <200 | <200 |
| Bacillus cereus | 2.8E + 06 | 2.8E + 06 |
| Candida albicans | <200 | <200 |
| Aspergillus niger | <200 | 1.1E + 05 |

The number of microorganisms, and especially of Aspergillus niger, after 7 days was determined. It was found that composition 1 afforded good antimicrobial protection, such as good antifungal protection.

### EXAMPLE 2

Shampoos of compositions 3, 4, and 5 below were prepared:

| Composition | 3 | 4 | 5 |
|---|---|---|---|
| Sodium lauryl ether sulfate | 20 g | 15 g | 20 g |
| containing 70% active material | | | |
| Cocamidopropyl betaine | 6.4 g | 6.4 g | 6.4 g |
| containing 37% active material | | | |
| Polyquaternium-10 | — | — | 0.5 g |
| Sodium chloride | 3.5 g | 3.8 g | — |
| Glycerol | 3 g | 4 g | 10 g |
| Hexylene glycol | 1 g | — | — |
| Fructose | 2 g | — | — |
| Sodium benzoate | — | — | 0.3 g |
| Salicylic acid | — | 0.3 g | — |
| Sodium dehydroacetate | 0.2 g | — | — |
| Water qs | 100 g | 100 g | 100 g |
| pH | 6.1 | 5.1 | 5.0 |
| Water activity | 0.95 | 0.95 | 0.95 |

1. A cosmetic and/or dermatological composition comprising, in a physiologically acceptable medium:
a) at least one surfactant;
b) at least one monocarboxylic acid chosen from benzoic acid, salicylic acid, sorbic acid, dehydroacetic acid, propionic acid, caproic acid, and salts thereof, wherein said at least one monocarboxylic acid is present in the composition in an amount ranging from 0.1% to 1% by weight, relative to the total weight of the composition; and
c) at least one polyol having a molecular mass of less than 500, wherein said at least one polyol is present in the composition in an amount ranging from 1 % to 20% by weight, relative to the total weight of the composition; wherein the water activity at 25° C. of the composition is less than or equal to 0.96.

2. The composition according to claim 1, wherein the composition is a haircare composition.

3. The composition according to claim 1, wherein the at least one monocarboxylic acid is chosen from benzoic acid, salicylic acid, sorbic acid, and dehydroacetic acid.

4. The composition according to claim 3, wherein the at least one monocarboxylic acid is chosen from benzoic acid, sorbic acid, and dehydroacetic acid.

5. The composition according to claim 1, wherein the monocarboxylic acid is present in the composition in an amount ranging from 0.15% to 0.5% by weight, relative to the total weight of the composition.

6. The composition according to claim 1, wherein the at least one polyol is chosen from sorbitol, glucose, fructose, xylose, trehalose, sucrose, maltose, lactose, polyethylene glycols, C₃-C₈ diols, and C₃-C₈ triols.

7. The composition according to claim 6, wherein the at least one polyol is chosen from glycerol and 1,2-propylene glycol.

8. The composition according to claim 7, wherein the at least one polyol is glycerol.

9. The composition according to claim 1, wherein the at least one polyol is present in the composition in an amount ranging from 1% to 10% by weight, relative to the total weight of the composition.

10. The composition according to claim 9, wherein the at least one polyol is present in the composition in an amount ranging from 4% to 10% by weight, relative to the total weight of the composition.

11. The composition according to claim 1, further comprising at least one additional salt other than the salt of monocarboxylic acid and of the at least one surfactant.

12. The composition according to claim 11, wherein the at least one additional salt is chosen from mineral salts, C₁-C₇ organic acid salts, organic salts of ammonium, and organic salts of a primary amine, optionally hydroxylated.

13. The composition according to claim 11, wherein the at least one additional salt is a salt of a metal chosen from Na, K, Li, Ca, Mg, Cu, and Zn.

14. The composition according to claim 11, wherein the at least one additional salt is chosen from sodium chloride, potassium chloride, lithium chloride, ammonium chloride, sodium sulphate, magnesium sulphate, sodium acetate, copper sulphate, and sodium citrate.

15. The composition according to claim 11, wherein the at least one additional salt is present in the composition in an amount ranging from 1% to 10% by weight, relative to the total weight of the composition.

16. The composition according to claim 15, wherein the at least one additional salt is present in the composition in an amount ranging from 1% to 5% by weight, relative to the total weight of the composition.

17. The composition according to claim 16, wherein the at least one additional salt is present in the composition in an amount ranging from 1% to 4% by weight, relative to the total weight of the composition.

18. The composition according to claim 11, wherein the at least one additional salt to at least one polyol mass ratio ranges from 1:1 to 1:10.

19. The composition according to claim 1, wherein the water activity at 25° C. ranges from 0.75 to 0.95.

20. The composition according to claim 1, wherein the pH of the composition is less than 7.

21. The composition according to claim 20, wherein the pH of the composition is less than 6.

22. The composition according to claim 1, wherein the physiologically acceptable medium comprises water or a mixture of water and at least one organic solvent chosen from C₁-C₄ alcohols.

23. The composition according to claim 1, further comprising at least one additive chosen from fragrances; screening agents; preserving agents; proteins; vitamins; nonionic, anionic, cationic, amphoteric, and zwitterionic polymers; mineral, plant, and synthetic oils; antidandruff agents; agents for preventing hair loss; dyes; pigments; and reducing agents.

24. The composition according to claim 1, wherein the composition is in the form of a shampoo.

25. A cosmetic and/or dermatological composition comprising, in a physiologically acceptable medium:
a) at least one surfactant;
b) at least one monocarboxylic acid chosen from benzoic acid, salicylic acid, sorbic acid, dehydroacetic acid, propionic acid, caproic acid, and salts thereof, wherein said at least one monocarboxylic acid is present in the composition in an amount ranging from 0.1% to 1% by weight, relative to the total weight of the composition; and
c) at least one polyol having a molecular mass of less than 500, wherein said at least one polyol is present in the composition in an amount ranging from 3% to 20% by weight, relative to the total weight of the composition.

26. The composition according to claim 25, wherein the at least one polyol is present in the composition in an amount ranging from 4% to 20% by weight, relative to the total weight of the composition.

27. A method for cosmetically treating the hair and/or the scalp, comprising applying to the hair and/or the scalp a composition comprising, in a physiologically acceptable medium:
a) at least one surfactant;
b) at least one monocarboxylic acid chosen from benzoic acid, salicylic acid, sorbic acid, dehydroacetic acid, propionic acid, caproic acid, and salts thereof, wherein said at least one monocarboxylic acid is present in the composition in an amount ranging from 0.1% to 1% by weight, relative to the total weight of the composition; and
c) at least one polyol having a molecular mass of less than 500, wherein said at least one polyol is present in the composition in an amount ranging from 1 % to 20% by weight, relative to the total weight of the composition; wherein the water activity at 25° C. of the composition is less than or equal to 0.96;
and optionally rinsing the hair and/or the scalp.

28. An antifungal cosmetic composition comprising, in a physiologically acceptable medium:
a) at least one surfactant;
b) at least one monocarboxylic acid chosen from benzoic acid, salicylic acid, sorbic acid, dehydroacetic acid, propionic acid, caproic acid, and salts thereof, wherein said at least one monocarboxylic acid is present in the composition in an amount ranging from 0.1% to 1% by weight, relative to the total weight of the composition; and
c) at least one polyol having a molecular mass of less than 500, wherein said at least one polyol is present in the composition in an amount ranging from 1% to 20% by weight, relative to the total weight of the composition; wherein the water activity at 25° C. of the composition is less than or equal to 0.96.

29. A method for treating disorders related to Aspergillus niger comprising, applying to hair or skin a dermatological composition comprising, in a physiologically acceptable medium:
a) at least one surfactant;
b) at least one monocarboxylic acid chosen from benzoic acid, salicylic acid, sorbic acid, dehydroacetic acid, propionic acid, caproic acid, and salts thereof, wherein said at least one monocarboxylic acid is present in the composition in an amount ranging from 0.1% to 1% by weight, relative to the total weight of the composition; and
c) at least one polyol having a molecular mass of less than 500, wherein said at least one polyol is present in the composition in an amount ranging from 1% to 20% by weight, relative to the total weight of the composition; wherein the water activity at 25° C. of the composition is less than or equal to 0.96.
